(19) United States
(12) Patent Application Publication
(10) Pub. No.: US 2022/0356152 A1
(43) Pub. Date: Nov. 10, 2022

(54) A PROCESS FOR PURIFYING CRUDE 4,4'-DICHLORODIPHENYL SULFONE

(71) Applicant: BASF SE, Ludwigshafen am Rhein (DE)

(72) Inventors: Stefan BLEI, Ludwigshafen am Rhein (DE); Jessica Nadine HAMANN, Ludwigshafen am Rhein (DE); Lukas Karl METZGER, Ludwigshafen am Rhein (DE); Christian SCHUETZ, Ludwigshafen am Rhein (DE); Jun GAO, Ludwigshafen am Rhein (DE); Frauke THRUN, Ludwigshafen am Rhein (DE); Andreas MELZER, Ludwigshafen am Rhein (DE); Michael Klemens MUELLER, Ludwigshafen am Rhein (DE)

(21) Appl. No.: 17/636,932

(22) PCT Filed: Aug. 20, 2020
(86) PCT No.: PCT/EP2020/073373

§ 371 Date: Feb. 21, 2022

(30) Foreign Application Priority Data
Aug. 27, 2019 (EP) 19193684.8

(51) Int. Cl. C07C 315/06

(52) U.S. Cl. CPC C07C 315/06

(57) ABSTRACT

The invention relates to a process for purifying crude 4,4′-dichlorodiphenyl sulfone comprising: (a) dissolving the crude 4,4′-dichlorodiphenyl sulfone which may contain water in an organic solvent in which 4,4′-dichlorodiphenyl sulfone has a solubility of 0.5 to 20% at 20° C. and optionally adding water to obtain a solution which comprises 4,4′-dichlorodiphenyl sulfone, the organic solvent and 1 to 30 wt % water based on the amount of 4,4′-dichlorodiphenyl sulfone and water; (b) cooling the solution to a temperature below the saturation point of 4,4′-dichlorodiphenyl sulfone to obtain a suspension comprising crystallized 4,4′-dichlorodiphenyl sulfone; (c) carrying out a solid-liquid separation to obtain residual moisture containing 4,4′-dichlorodiphenyl sulfone and a mother liquor; (d) washing the residual moisture containing 4,4′-dichlorodiphenyl sulfone with an organic solvent in which 4,4′-dichlorodiphenyl sulfone has a solubility of 0.5 to 20% at 20° C.; (e) optionally repeating steps (b) to (d); (f) drying the 4,4′-dichlorodiphenyl sulfone.

The invention relates to a process for purifying crude 4,4′-dichlorodiphenyl sulfone by dissolving the 4,4′-dichlorodiphenyl sulfone in a C₁ to C₃ alcohol, forming a suspension, carrying out a solid-liquid separation of the suspension and washing the obtained moist 4,4′-dichlorodiphenyl sulfone with a C₁ to C₃ alcohol.

4,4′-dichlorodiphenyl sulfone (in the following DCDPS) is used for example as a monomer for preparing polymers like polyether sulfone or polysulfone or as an intermediate of pharmaceuticals, dyes and pesticides.

DCDPS for example is produced by oxidation of 4,4′-dichlorodiphenyl sulfoxide which can be obtained by a Friedel-Crafts reaction of thionyl chloride and chlorobenzene as starting materials in the presence of a catalyst, for example aluminum chloride.

CN-A 108047101, CN-A 102351758, CN-B 104402780 and CN-A 104557626 disclose a two-stage process in which in a first stage a Friedel-Crafts acylation reaction is carried out to produce 4,4′-dichlorodiphenyl sulfoxide and in a second stage the 4,4′-dichlorodiphenyl sulfoxide is oxidized to obtain DCDPS using hydrogen peroxide as oxidizing agent. The oxidation reaction thereby is carried out in the presence of acetic acid. Such a process in which 4,4′-dichloro-diphenyl sulfoxide is produced in a first stage and DCDPS is obtained in a second stage using hydrogen peroxide in excess and acetic acid as solvent also is described in SU-A 765262.

Further processes for obtaining DCDPS by reacting chlorobenzene and thionyl chloride in a Friedel-Crafts reaction in a first stage to obtain 4,4′-dichlorodiphenyl sulfoxide and to oxidize the 4,4′-dichlorodiphenyl sulfoxide in a second stage using hydrogen peroxide as oxidizing agent and dichloromethane or dichloropropane as solvent are disclosed in CN-A 102351756 and CN-A 102351757.

A process for producing an organic sulfone by oxidation of the respective sulfoxide in the presence of at least one peroxide is disclosed in WO-A 2018/007481. The reaction thereby is carried out in a carboxylic acid as solvent, the carboxylic acid being liquid at 40° C. and having a miscibility gap with water at 40° C. and atmospheric pressure.

DE-A 37 04 931 describes a process for isolating DCDPS from a mixture which comprises 0.5 to 20 wt % 2,4′-dichlorodiphenyl sulfone and 0.5 to 30 wt % 3,4′-dichlorodiphenyl sulfone by mixing with an alkanol, cooling and separating the precipitated DCDPS.

In all of these processes the DCDPS containing reaction product is cooled after the reaction is completed to precipitate solid DCDPS and to separate the solid DCDPS from the mixture. After separation, in those processes where the reaction is carried out in a carboxylic acid as solvent, the solid DCDPS is washed with water.

However, due to the reaction process, usually even the washed DCDPS still may contain remainders of the carboxylic acid, 4,4′-dichlorodiphenyl sulfoxide and isomers.

Therefore, it is an object of the present invention to provide a process to further purify the DCDPS.

This object is achieved by a process for purifying crude 4,4′-dichlorodiphenyl sulfone comprising:
(a) dissolving the crude DCDPS which may contain water in an organic solvent in which DCDPS has a solubility of 0.5 to 20% at 20° C. and optionally adding water to obtain a solution which comprises DCDPS, the organic solvent and 1 to 30 wt % water based on the amount of DCDPS and water;
(b) cooling the solution to a temperature below the saturation point of DCDPS to obtain a suspension comprising crystallized 4,4′-dichlorodiphenyl sulfone;
(c) carrying out a solid-liquid separation to obtain residual moisture containing DCDPS and a mother liquor;
(d) washing the residual moisture containing DCDPS with an organic solvent in which DCDPS has a solubility of 0.5 to 20% at 20° C.;
(e) optionally repeating steps (b) to (d);
(f) drying the 4,4′-dichlorodiphenyl sulfone.

The solubility of DCDPS in the organic solvent is defined as

S=m_{DCDPS}/mₛₒₗᵥ·100 [%]

with m_{DCDPS}=amount of DCDPS in kg
mₛₒₗᵥ=amount of solvent in kg.

The crude DCDPS to be purified for example emanates from an oxidation reaction of 4,4′-dichlorodiphenyl sulfoxide (in the following termed as DCDPSO) with an oxidizing agent, for example hydrogen peroxide in a solvent. After completing the reaction, the resulting reaction mixture usually is washed with water. The thus produced crude DCDPS may still contain from 0.1 to 0.9 wt % of the solvent, for example a carboxylic acid, 0.001 to 0.1 wt % DCDPSO and 0.01 to 0.3 wt % isomers of the DCDPS, for example 2,2′-dichlorodiphenyl sulfone, 3,4′-dichloro-diphenyl sulfone or 2,4′-dichlorodiphenyl sulfone. Depending on the oxidation reaction, the carboxylic acid for example is n-hexanoic acid, n-heptanoic acid or a mixture thereof. The amount of isomers thereby mainly depends on the purity of the DCDPSO. All amounts of the impurities contained in the crude DCDPS are based on the total amount of crude DCDPS. Further, the crude DCDPS still may contain water which was not removed by a solid-liquid separation, for example a filtration, following the washing steps. The water content in the crude DCDPS may be in the range from 1 to 30 wt %, preferably in the range from 2 to 25 wt % and particularly in the rage from 3 to 20 wt %.

By the inventive purification process it was possible to further reduce the impurities in the DCDPS and to achieve a DCDPS which contains less than 0.3 wt % isomers, less than 10 ppm 4,4′-dichlorodiphenyl sulfoxide, particularly less than 2 ppm 4,4′-dichlorodiphenyl sulfoxide and less than 200 ppm, particularly less than 100 ppm carboxylic acid, particularly n-hexanoic acid and/or n-heptanoic acid, each based on the total amount of dry DCDPS.

Surprisingly it has shown that when the solution obtained in (a) comprises water in an amount from 1 to 30 wt %, preferably from 2 to 25 wt % and particularly from 3 to 20 wt %, each based on the amount of water and DCDPS, the amount of undesired isomers, particularly 2,4′-dichlorodiphenyl sulfone and 3,4′-dichlorodiphenyl sulfone, can be further reduced. The presence of water in the solution has the further advantage that also at least a majority of the carboxylic acid and DCDPSO contained in the crude DCDPS can be removed.

Further, drying of the filter cake for removing water before dissolving the crude DCDPS in the organic solvent can be omitted.

To purify the crude DCDPS, at first crude solid DCDPS which may contain residual moisture, for example water from washing the DCDPS after completing the reaction, is mixed with the organic solvent in which DCDPS has a solubility of 0.5 to 20% at 20° C. (in the following termed as organic solvent). By mixing the crude solid DCDPS with the organic solvent, a suspension forms and the crude solid DCDPS starts to dissolve. If the crude DCDPS does not contain water or if the water content in the crude DCDPS is below 1 wt %, water is added to the suspension. If water is added to the suspension, it is possible to use a liquid mixture comprising the organic solvent and water or to add the water separately. The water can be added simultaneously with the organic solvent, before adding the organic solvent or after finishing the addition of the organic solvent. However, particularly preferably, crude DCDPS is used which contains water in an amount from 1 to 30 wt %, more preferred from 2 to 25 wt % and particularly from 3 to 20 wt %.

For mixing the crude solid DCDPS with the organic solvent, in a batch process it is preferred, to provide the organic solvent in a suitable vessel and to add the crude solid DCDPS in the form of crystallized pellets or as a powder to the organic solvent. The mixing also can be carried out continuously. For a continuous mixing, the crude solid DCDPS is fed continuously into a mixing device and the organic solvent is added continuously in appropriate amounts. The mixing device for example also for continuous mixing may be a mixing vessel. In this case the suspension formed in the vessel also is continuously withdrawn. The crude solid DCDPS preferably directly is fed from a solid/liquid separation, for example a filtration, into the mixing device. If the crude DCDPS does not contain a sufficient amount of water, in the batch process it is preferred to add water to the organic solvent in the vessel before adding the crude solid DCDPS or to mix the crude solid DCDPS with water before adding into the vessel. In a continuous process it is also possible to add the water to the crude DCDPS before mixing with the organic solvent or alternatively to either add a mixture of organic solvent and an appropriate amount of water or to add the organic solvent and the water separately into the mixing device.

Independently of being carried out continuously or batchwise, by mixing in this order, it is avoided that the crude solid DCDPS agglomerates and forms a block which is lengthy and difficult to dissolve. The ratio of organic solvent to DCDPS preferably is in the range from 1.3 to 6, more preferred in the range from 1.5 to 4 and particularly in the range from 1.8 to 3. This ratio of organic solvent to DCDPS allows a complete dissolution of the DCDPS in the organic solvent by using the lowest possible amount of organic solvent.

To support dissolving the crude solid DCDPS in the organic solvent, the suspension comprising the crude solid DCDPS, the organic solvent and the water is heated. Preferably, the suspension is heated to a temperature in the range from 90 to 120° C., particularly in a range from 100 to 110° C. To avoid evaporation of the organic solvent during the heating of the suspension, the heating preferably is carried out at elevated pressure. Preferably, during heating for supporting dissolving the crude solid DCDPS in the organic solvent, the pressure is set to 2 to 10 bar(abs), more preferred to 3 to 5 bar(abs) and particularly to 3.5 to 4.5 bar(abs).

After completing dissolving the DCDPS in (a), the thus produced suspension is cooled to a temperature below the saturation point of DCDPS to obtain a suspension comprising crystallized DCDPS in a mother liquor comprising organic solvent and water (in the following termed as liquid phase). Due to cooling the suspension, the DCDPS starts to crystallize again. This new crystallization of the DCDPS in the mother liquor has the advantage, that impurities which may have been comprised in the crude DCDPS remain solved in the mother liquor and the crystals newly formed by cooling have a higher purity. Dissolving the DCDPS in the organic solvent and optionally the water to obtain the solution is completed when at least 90% of the DCDPS are dissolved. Particularly preferably, dissolving the DCDPS in the organic solvent is completed when all of the DCDPS is dissolved.

The saturation point denotes the temperature of the solution at which DCDPS starts to crystallize. This temperature depends on the concentration of the DCDPS in the solution. The lower the concentration of DCDPS in the solution, the lower is the temperature at which crystallization starts.

To avoid a too fast crystal growth by which impurities solved in the mother liquor would be incorporated into the newly formed crystals, it is preferred to cool the solution in (b) with a multi-step cooling rate of initially from 3 to 15 K/h for 0.5 to 3 h more preferred from 0.5 to 2 h and later on from 10 to 40 K/h, more preferred with a cooling rate from 15 to 30 K/h and particularly with a cooling rate from 18 to 25 K/h until a predefined end temperature is reached. Besides the preferred multi-step cooling, a one-step cooling with a cooling rate in the range from 10 to 30 K/h until the end temperature is reached, also is possible.

The lower the temperature to which the solution is cooled, the lower is the amount of DCDPS still solved in the mother liquor. On the other hand, the efforts needed for cooling increase with decreasing temperature. Therefore, the solution preferably is cooled in (b) to a temperature in the range from −10 to 25° C., more preferred to a temperature in the range from 0 to 20° C. and particularly to a temperature in the range from 3 to 12° C. Cooling to a temperature in such a range has the advantage that the stage yield in regard to the necessary efforts is optimized. This has the additional effect that waste streams of the overall process can be minimized.

Cooling of the solution for crystallizing DCDPS can be carried out in any crystallization apparatus which allows cooling of the solution. Such apparatus for example are apparatus with surfaces that can be cooled like a vessel or a tank with cooling jacket, cooling coils or cooled baffles like so called “power baffles”.

Cooling of the solution for crystallization of the DCDPS can be performed either continuously or batchwise. To avoid precipitation and fouling on cooled surfaces, it is preferred to carry out the cooling in a gastight closed vessel by
(i) reducing the pressure of the solution to a pressure at which the organic solvent starts to evaporate;
(ii) condensing the evaporated organic solvent by cooling;
(iii) mixing the condensed organic solvent with the solution to obtain the suspension.

This process allows for cooling the solution without cooled surfaces onto which crystallized DCDPS accumulates and forms a solid layer. This enhances the efficiency of the cooling process. Also, additional efforts to remove this solid layer can be avoided. Therefore, it is particularly preferred to use a gastight closed vessel without cooled surfaces.

In this process for cooling in a gastight closed vessel it cannot be excluded that besides the organic solvent also a part of the water evaporates. Therefore, when the term “organic solvent” is used in the description of evaporation steps and condensation steps in the cooling process, the skilled person appreciates that the organic solvent also may comprise water.

To avoid precipitation of the crystallized DCDPS it is further preferred to agitate the suspension in the crystallization apparatus. Therefore, suitable apparatus is for example a stirred tank or a draft-tube crystallizer. If the crystallization is carried out in a stirred tank, any stirrer can be used.

The specific power input into the crystallizer by the stirrer preferably is in the range from 0.2 to 0.5 W/kg, more preferred in the range from 0.2 to 0.35 W/kg. Preferably, a stirrer type is used which leads to a rather homogeneous power input without high gradients concerning local energy dissipation.

The pressure reduction (i) to evaporate the organic solvent can be either stepwise or continuous. If the pressure reduction is stepwise, it is preferred to hold the pressure in one step until a predefined rate in temperature decrease can be observed, particularly until the predefined rate is “0” which means that no further temperature decrease occurs. After this state is achieved, the pressure is reduced to a next predefined pressure value of the following pressure step.

In this case the steps for reducing the pressure all can be the same or can be different. If the pressure is reduced in different steps, it is preferred to reduce the size of the steps with decreasing pressure. The pressure steps depend on the solvent used. Particularly preferably, the stepwise pressure reduction is carried out in such a way that in each step the temperature is reduced by 1 to 10 K, more preferred by 1 to 7 K and particularly by 1 to 3 K.

If the pressure reduction is continuously, the pressure reduction can be for example linearly, hyperbolic, parabolic or in any other shape, wherein it is preferred for a non-linear decrease in pressure to reduce the pressure in such a way that the pressure reduction decreases with decreasing pressure.

The cooling (b) of the solution can be carried out batchwise, semi-continuously or continuously.

If the cooling and thus the crystallization of DCDPS is performed batchwise it is preferred to carry out condensing (ii) and mixing (iii) during the pressure reduction (i). Thereby, it is particularly preferred to continuously reduce the pressure in step (i) until the temperature in the gas-tight closed vessel reaches the predefined value in the range from −10 to 25° C., preferably in the range from 0 to 20° C. and particularly in the range from 3 to 12° C. At these predefined temperatures the pressure in the gastight closed vessel typically is in the range from 10 to 400 mbar(abs), more preferred in the range from 10 to 200 mbar(abs) and particularly in the range from 30 to 80 mbar(abs). After the predefined temperature value is reached, pressure reduction is stopped and then the gastight closed vessel is vented until ambient pressure is reached. The temperature profile in the gastight closed vessel preferably is selected such that the solution is subjected to a constant supersaturation. These conditions can be achieved by adapting the cooling profile while keeping the temperature below the saturation temperature at the respective concentration of DCDPS in the solution. In detail the adapted cooling profile is chosen, based on phase equilibria, mass of crystal nuclei, and initial size of the crystal nuclei. Further, to adapt the cooling profile, constant grow rates are assumed. To determine the data for adapting the cooling profile, for example turbidity probes, refractive index probes or ATR-FTIR-probes can be used. The temperature profile and/or pressure profile for example can be stepwise, linear or progressive.

To reduce the solubility of the DCDPS and thus increase the yield of solidified DCDPS it is necessary to shift the saturation point. This is possible by continuously reducing the amount of organic solvent at a constant temperature, for example by evaporating organic solvent, or by cooling the solution at constant concentration or by a hybrid procedure by reducing the amount of organic solvent by evaporation followed by reducing the temperature. For reducing the solubility of DCDPS in the solution and to improve the crystallization, it is further possible to additionally add at least one drowning-out agent, for example water.

After reaching ambient pressure the suspension comprising particulate 4,4′-dichlorodiphenyl sulfone in the organic solvent (in the following termed as “suspension”) which formed in the gas-tight closed vessel by the cooling is withdrawn and fed into the solid-liquid-separation (c).

If the cooling and thus the crystallization of DCDPS is performed continuously, it is preferred to operate the cooling and crystallization stepwise in at least two steps, particularly in two to three steps. If the cooling and crystallization is carried out in two steps, in a first step the solution preferably is cooled to a temperature in the range from 70 to 110° C. and in a second step preferably to a temperature in the range from −10 to 25° C. If the cooling is operated in more than two steps, the first step preferably is operated at a temperature in the range from 70 to 110° C. and the last step at a temperature in the range from −10 to 25° C. The additional steps are operated at temperatures between these ranges with decreasing temperature from step to step. If the cooling and crystallization is performed in three steps, the second step for example is operated at a temperature in the range from 20 to 70° C.

As in the batchwise process, the temperature in the continuously operated process can be set by using apparatus for cooling and crystallization having surfaces to be cooled, for example a cooled jacket, cooling coils or cooled baffles like so called “power baffles”. To establish the at least two steps for cooling and crystallization, for each step at least one apparatus for cooling and crystallization is used. To avoid precipitation of DCDPS, also in the continuous process it is preferred to reduce the temperature by reducing the pressure in the apparatus for cooling and crystallization wherein the apparatus for cooling and crystallization preferably are gastight closed vessels. Further suitable apparatus for cooling and crystallization for example are agitated-tank crystallizers, draft-tube crystallizers, horizontal crystallizers, forced-circulation crystallizers or Oslo-crystallizers. The pressure which is set to achieve the required temperature corresponds to the vapor pressure of the solution. Due to the pressure reduction, low boilers, particularly organic solvent, evaporate. The evaporated low boilers are cooled to condense, and the condensed low boilers are returned into the respective apparatus for cooling and crystallization by which the temperature is set.

If the cooling and crystallization is carried out continuously, a stream of the suspension is continuously withdrawn from the apparatus for cooling and crystallization. The suspension then is fed into the solid-liquid-separation (c). To keep the liquid level in the apparatus for cooling and crystallization within predefined limits fresh solution comprising DCDPS and organic solvent can be fed into the apparatus in an amount corresponding or essentially corresponding to the amount of suspension withdrawn from the apparatus. The fresh solution either can be added continuously or batchwise each time a minimum liquid level in the apparatus for cooling and crystallization is reached.

Independently of being carried out batchwise or continuously, crystallization preferably is continued until the solids content in the suspension in the last step of the crystallization is in the range from 5 to 50 wt %, more preferred in the range from 5 to 40 wt % and particularly in the range from 15 to 40 wt %, based on the mass of the suspension.

Even though the cooling and crystallization can be carried out continuously or batchwise, it is preferred to carry out the cooling and crystallization batchwise and particularly to cool the solution by reducing the pressure according to the above described process comprising steps (i) to (iii) to avoid precipitation of crystallized DCDPS on cooled surfaces of an apparatus for cooling and crystallization. Batchwise cooling and crystallization allows a higher flexibility in terms of operating window and crystallization conditions and is more robust against variations in process conditions.

Independently of whether the cooling and crystallization is performed continuously or batchwise, the solid-liquid-separation (c) can be carried out either continuously or batchwise, preferably continuously.

If the cooling and crystallization is carried out batchwise and the solid-liquid-separation is carried out continuously at least one buffer container is used into which the suspension withdrawn from the apparatus used for cooling and crystallization is filled. For providing the suspension a continuous stream is withdrawn from the at least one buffer container and fed into a solid-liquid-separation apparatus. The volume of the at least one buffer container preferably is such that each buffer container is not totally emptied between two filling cycles in which the contents of the apparatus for cooling and crystallization is fed into the buffer container. If more than one buffer container is used, it is possible to fill one buffer container while the contents of another buffer container are withdrawn and fed into the solid-liquid-separation. In this case the at least two buffer containers are connected in parallel. The parallel connection of buffer containers further allows filling the suspension into a further buffer container after one buffer container is filled. An advantage of using at least two buffer containers is that the buffer containers may have a smaller volume than only one buffer container. This smaller volume allows a more efficient mixing of the suspension to avoid sedimentation of the crystallized DCDPS. To keep the suspension stable and to avoid sedimentation of solid DCDPS in the buffer container, it is possible to provide the buffer container with a device for agitating the suspension, for example a stirrer, and to agitate the suspension in the buffer container. Agitating preferably is operated such that the energy input by stirring is kept on a minimal level, which is high enough to suspend the crystals but prevents them from breakage. For this purpose, the energy input preferably is preferably in the range from 0.2 to 0.5 W/kg, particularly in the range from 0.25 to 0.4 W/kg and a tip speed of the stirrer below 3 m/s.

If the cooling and crystallization and the solid-liquid-separation are carried out batchwise the contents of the vessel for cooling and crystallization directly can be fed into a solid-liquid-separation apparatus as long as the solid-liquid separation apparatus is large enough to take up the whole contents of the vessel for cooling and crystallization. In this case it is possible to omit the buffer container. It is also possible to omit the buffer container when cooling and crystallization and the solid-liquid-separation are carried out continuously. In this case also the suspension directly is fed into the solid-liquid-separation apparatus. If the solid-liquid separation apparatus is too small to take up the whole contents of the vessel for cooling and crystallization, also for batchwise operation at least one additional buffer container is necessary to allow to empty the crystallization apparatus and to start a new batch.

If the cooling and crystallization are carried out continuously and the solid-liquid-separation is carried out batchwise the suspension withdrawn from the cooling and crystallization apparatus is fed into the buffer container and each batch for the solid-liquid-separation is withdrawn from the buffer container and fed into the solid-liquid-separation apparatus.

The solid-liquid-separation for example comprises a filtration, centrifugation or sedimentation. Preferably, the solid-liquid-separation is a filtration. In the solid-liquid-separation mother liquor is removed from the solid DCDPS and residual moisture containing DCDPS (in the following also termed as “moist DCDPS”) is obtained. If the solid-liquid-separation is a filtration, the moist DCDPS is called “filter cake”.

Independently of carried out continuously or batchwise, the solid-liquid-separation preferably is performed at ambient temperature or temperatures below ambient temperature, preferably at a temperature in the range from 0 to 10° C. It is possible to feed the suspension into the solid-liquid-separation apparatus with elevated pressure for example by using a pump or by using an inert gas having a higher pressure, for example nitrogen. If the solid-liquid-separation is a filtration and the suspension is fed into the filtration apparatus with elevated pressure the differential pressure necessary for the filtration process is realized by setting ambient pressure to the filtrate side in the filtration apparatus. If the suspension is fed into the filtration apparatus at ambient pressure, a reduced pressure is set to the filtrate side of the filtration apparatus to achieve the necessary differential pressure. Further, it is also possible to set a pressure above ambient pressure on the feed side of the filtration apparatus and a pressure below ambient pressure on the filtrate side or a pressure below ambient pressure on both sides of the filter in the filtration apparatus, wherein also in this case the pressure on the filtrate side must be lower than on the feed side. Further, it is also possible to operate the filtration by only using the static pressure of the liquid layer on the filter for the filtration process. Preferably, the pressure difference between feed side and filtrate side and thus the differential pressure in the filtration apparatus is in the range from 100 to 6000 mbar(abs), more preferred in the range from 300 to 2000 mbar(abs) and particularly in the range from 400 to 1500 mbar(abs), wherein the differential pressure also depends on the filters used in the solid-liquid-separation (c).

To carry out the solid-liquid-separation (c) any solid-liquid-separation apparatus known by the skilled person can be used. Suitable solid-liquid-separation apparatus are for example an agitated pressure nutsche, a rotary pressure filter, a drum filter, a belt filter or a centrifuge. The pore size of the filters used in the solid-liquid-separation apparatus preferably is in the range from 1 to 1000 μm, more preferred in the range from 10 to 500 pm and particularly in the range from 20 to 200 μm.

Particularly preferably, cooling and crystallization is carried out batchwise and the solid-liquid-separation is operated continuously.

To further purify the DCDPS and to remove impurities from the surface of the crystallized DCDPS and which may be contained in the remaining organic solvent in the moist DCDPS as well as non-crystallized DCDPS, the moist DCDPS is washed with a an organic solvent in which DCDPS has a solubility of 0.5 to 20% at 20° C. (in the following also termed as organic solvent).

The amount of organic solvent used for washing preferably is chosen such that the impurities and the non-crystallized DCDPS are removed from the moist DCDPS. Preferably, the amount of organic solvent used for washing is in a range from 0.3 to 3 kg per kg moist DCDPS, more preferred in a range from 0.5 to 2 kg per kg moist DCDPS and particularly in a range from 0.8 to 1.5 kg per kg moist DCDPS. The lower the amount of organic solvent for washing, the lower are the efforts for recycling the organic solvent and to reuse it in the process cycle, but the less the amount of organic solvent, the less is the washing efficiency regarding carboxylic acids, particularly n-hexanoic acid and/or n-heptanoic acid, and remaining 4,4′-DCDPSO and isomers of 4,4′-DCDPS.

The solid-liquid separation and the washing of the moist DCDPS can be carried out in the same apparatus or in different apparatus. If the solid-liquid separation is a filtration it is possible to carry out the following washing of the filter cake in the filtration apparatus, independently of whether the filtration is operated continuously or batchwise. After washing, the filter cake is removed and dried to obtain dry DCDPS as product.

Besides carrying out filtration and washing of the filter cake in one apparatus, it is also possible to withdraw the filter cake from the filtration apparatus and wash it in a subsequent washing apparatus. If the filtration is carried out on a belt filter, it is possible to convey the filter cake on the filter belt into the washing apparatus. For this purpose, the filter belt is designed in such a way that it leaves the filtration apparatus and enters into the washing apparatus. Besides transporting the filter cake on a filter belt from the filtration apparatus into the washing apparatus it is also possible to collect the filter cake with a suitable conveyor and feed the filter cake from the conveyor into the washing apparatus. If the filter cake is withdrawn from the filtration apparatus with a suitable conveyor the filter cake can be withdrawn from the filtration apparatus as a whole, or in smaller pieces such as chunks or pulverulent. Chunks for instance arise if the filter cake breaks when it is withdrawn from the filtration apparatus. To achieve a pulverulent form, the filter cake usually must be comminuted. Independently from the state of the filter cake, for washing the filter cake is brought into contact with the organic solvent. For example, the filter cake can be put on a suitable tray in the washing apparatus and the organic solvent flows through the tray and the filter cake. Further it is also possible to break the filter cake into smaller chunks or particles and to mix the chunks or particles with the organic solvent. Subsequent the thus produced mixture of chunks or particles of the filter cake and the organic solvent is filtrated to remove the organic solvent. If the washing is carried out in a separate washing apparatus, the washing apparatus can be any suitable apparatus. Preferably the washing apparatus is a filter apparatus which allows to use a smaller amount of organic solvent and to separate the organic solvent from the solid DCDPS in only one apparatus. However, it is also possible to use for example a stirred tank as washing apparatus. In this case it is necessary to separate the organic solvent from the washed DCDPS in a following step, for example by filtration or centrifugation.

If the solid-liquid-separation (c) is carried out by centrifugation, depending on the centrifuge it might be necessary to use a separate washing apparatus for washing the moist DCDPS. However, usually a centrifuge can be used which comprises a separation zone and a washing zone or the washing can be carried out after centrifuging in the centrifuge.

Washing of the moist DCDPS preferably is operated at ambient temperature. It is also possible to wash the moist DCDPS at temperatures different to ambient temperature, for instance above ambient temperature. If the washing is carried out in the filtration apparatus, for washing the filter cake a differential pressure must be established. This is possible for example by feeding the organic solvent for washing the filter cake at a pressure above ambient pressure and withdraw the organic solvent after passing the filter cake at a pressure below the pressure at which the organic solvent is fed, for example at ambient pressure. Further it is also possible to feed the organic solvent for washing the filter cake at ambient pressure and withdraw the organic solvent and the water after passing the filter cake at a pressure below ambient pressure.

The mother liquor obtained in the solid-liquid separation and the organic solvent used for washing still may contain non-crystallized DCDPS. To increase the yield of purified DCDPS in the process and to reduce the amount of organic solvent to be disposed, preferably, at least a part of the mother liquor and optionally the organic solvent used for washing are worked up by distillation.

By working up at least a part of the mother liquor and optionally the organic solvent used for washing, it is possible to withdraw at least a part of the DCDPS still solved in the organic solvent as a high boiler and to recycle at least a part of the high boilers either into the purifying process or into a process step upstream the purifying process to obtain the DCDPS as product and to increase the yield. Further, the organic solvent which is purified in the distillation and obtained as low boiler can be recycled into the purifying step either as organic solvent for solving the DCDPS or as organic solvent for washing the DCDPS. If the organic solvent is used for washing the DCDPS, it must fulfil predefined purity requirements. For being used for washing, the organic solvent preferably contains less than 0.05 wt % impurities, more preferred less than 0.03 wt % impurities and particularly less than 0.015 wt % impurities, each based on the total mass of the organic solvent.

The organic solvent which is used for dissolving the DCDPS preferably has the same purity as the organic solvent used for washing, however, it is also possible to use an organic solvent which is less pure for dissolving the DCDPS. To achieve a product which has the above identified purity, the organic solvent used for dissolving the DCDPS preferably contains less than 0.05 wt % impurities, more preferred less than 0.03 wt % impurities and particularly less than 0.015 wt % impurities, each based on the total mass of the organic solvent.

Particularly preferably, the amount of mother liquor and optionally organic solvent used for washing which is worked up by distillation is in a range from 50 to 100 wt %, more preferred in a range from 70 to 100 wt % and particularly in a range from 90 to 100 wt %, each based on the total amount of mother liquor and organic solvent used for washing.

The organic solvent which is used for dissolving the DCDPS and the organic solvent which is used for washing the moist DCDPS preferably additionally is chosen such that the solubility of DCDPS at the boiling point of the organic solvent is up to 100%. Suitable organic solvents for example are symmetric or asymmetric, branched or linear ethers, for example diethyl ether or methyl tert-butyl ether, substituted or unsubstituted aromatic solvents like toluene, monochloro-benzene or benzene, low molecular carboxylic acids, particularly C₁ to C₃ carboxylic acids or low molecular alcohols, particularly C₁ to C₃ alcohols. Preferably, the organic solvent is methanol, ethanol, isopropanol, acetone, methyl tert-butyl ether, acetic acid, toluene, ethyl acetate or monochlorobenzene. Particularly preferably, the organic solvent is a C₁ to C₃ alcohol, particularly methanol, ethanol or isopropanol. Most preferred the organic solvent is methanol.

For dissolving the DCDPS and for washing different organic solvents can be used. However, it is particularly preferred to use the same organic solvent to dissolve the DCDPS for obtaining the solution and for washing the moist DCDPS. Using the same organic solvent allows to work up the mother liquor and the organic solvent used for washing together, whereas to allow reuse of the organic solvents if different organic solvents are used for dissolving the DSCDPS and for washing the moist DCDPS each organic solvent has to be worked up separately to avoid mixing of the organic solvents.

If the DCDPS after solid-liquid separation and washing still contains a too large amount of impurities, it is possible to repeat the process steps (a) to (d).

To achieve a dry product, after washing the DCDPS which still contains organic solvent is dried. Drying can be carried out in any dryer which can be used for drying particulate substances. A suitable dryer for example is a paddle dryer, a tumble dryer, or any other type of contact dryer or a fluidized bed dryer. Further, also a combination of at least two of the dryer types can be used.

Drying preferably is carried out using a contact dryer with a wall temperature in the range from 105 to 140° C., more preferred in a range from 110 to 135° C. and particularly in a range from 120 to 135° C. By drying the DCDPS at such a temperature, coloring of the DCDPS can be avoided. Drying preferably is continued for 90 to 600 min, more preferred for 180 to 350 min and particularly for 200 to 300 min.

To support the drying process and to avoid damaging the product for example by oxidation, drying in the dryer preferably is carried out in an inert atmosphere. The inert atmosphere is achieved by feeding an inert gas into the dryer. The inert gas preferably is nitrogen, carbon dioxide or a noble gas, for example argon. Particularly preferably, the inert gas is nitrogen.

To allow reusing the organic solvent which is removed from the DCDPS during drying by evaporation, the evaporated organic solvent is condensed by cooling. If an inert gas is fed into the dryer, usually the inert gas and the evaporated organic solvent are withdrawn together from the dryer. In this case, in the condenser the condensed organic solvent is separated from the inert gas. The organic solvent for example can be reused for producing the solution or for washing the DCDPS.

By drying the DCDPS at these conditions, a final product can be achieved which contains less than 400 ppm organic solvent.

After drying, the DCDPS can be cooled down to enable further handling, for example packing in Bigbags for storage or transport. Suitable coolers for cooling the dried DCDPS can be screw coolers, paddle coolers or other bulk coolers or fluidized bed coolers.

An illustrative embodiment of the invention is shown in the figure and explained in more detail in the following description.

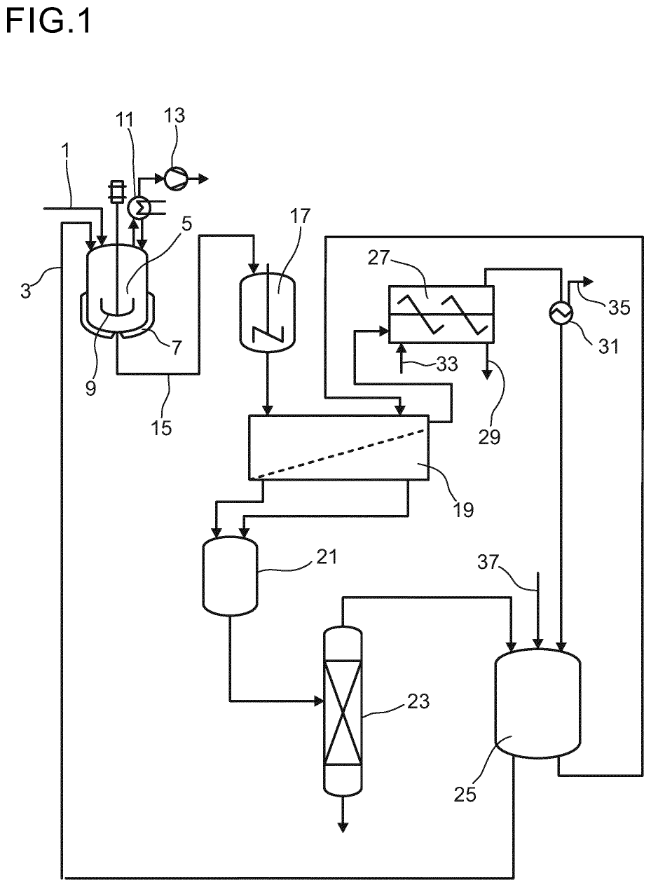

FIG. 1 shows a flow diagram of an embodiment of the inventive process.

In FIG. 1 the process for purifying crude DCDPS is shown in a flow diagram.

To purify crude DCDPS, particulate crude DCDPS 1 which preferably contains 1 to 30 wt % water and an organic solvent 3, preferably methanol, are fed into a gastight closed vessel 5. In the gastight closed vessel 5 the particulate crude DCDPS 1 is solved in the organic solvent 3. To support solving the particulate crude DCDPS 1 in the organic solvent 3, the mixture of particulate crude DCDPS and organic solvent in the gastight closed vessel 5 is heated to a temperature in the range from 90 to 120° C. For heating the mixture, the gastight closed vessel 5 is equipped with a double jacket 7 which can be flown through by a heating medium, for example a thermal oil or steam. For supporting solving the crude DCDPS in the organic solvent, further an agitating means 9 is comprised for mixing the crude DCDPS and the organic solvent. The agitating means can be for example a stirrer. After completing solving the crude DCDPS in the organic solvent, the thus produced solution is cooled to recrystallize the DCDPS. If the particulate crude DCDPS 1 does not contain a sufficient amount of water, additionally water is fed into the gastight closed vessel 5 to achieve a solution which also contains 1 to 30 wt % water based on the amount of DCDPS and water.

For cooling the solution, the pressure in the gastight closed vessel 5 is reduced. Due to the pressure reduction, the organic solvent starts to evaporate. The evaporated organic solvent is withdrawn from the gastight closed vessel 5 and flows into a condenser 11. In the condenser 11, the vaporous organic solvent is cooled and condenses. The thus condensed and cooled organic solvent is returned into the gastight closed vessel 5. By the pressure reduction and the resulting evaporating and condensing the organic solvent, the solution in the gastight closed vessel 5 is cooled until a temperature in the range from 0 to 25° C. is achieved. Due to cooling the solution, the DCDPS crystallizes in the solution and a suspension is formed. To keep the crystallized DCDPS in the suspension and to avoid fouling on the surfaces of the gastight closed vessel 5, the suspension which forms in the gastight closed vessel 5 is mixed with the agitating means 9. To reduce the pressure in the gastight closed vessel, for example a vacuum pump 13 can be used which is arranged downstream the condenser 11. Evaporated organic solvent which is pumped out of the gastight closed vessel 5 can be condensed and collected or disposed.

After the desired temperature in the gastight closed vessel 5 is achieved, the suspension formed in the gastight closed vessel 5 is withdrawn via line 15 and fed into a buffer container 17. From the buffer container 17, the suspension is fed into a filtration apparatus 19. By using the buffer container 17 it is possible to carry out the crystallization in the gastight closed vessel 5 batchwise and the filtration in the filtration apparatus 19 continuously. However, also for the crystallization and the filtration being carried out batchwise it is preferred to use the buffer container 17, to allow use of a filtration apparatus 19 which has a different capacity than the gas-tight closed vessel 5. This allows to use a gastight closed vessel 5 and a filtration apparatus 19 which each are optimized regarding throughput and energy consumption. In the filtration apparatus 19 the crystallized DCDPS is separated from the mother liquor which contains the organic solvent, water, non-crystallized DCDPS and impurities. The mother liquor is withdrawn from the filtration apparatus 19 and collected in an organic solvent collecting tank 21. After being separated from the mother liquor, the residual moisture containing DCDPS is washed with organic solvent. In the embodiment shown in the figure, the washing also is carried out in the filtration apparatus. After being used for washing, the organic solvent also is collected in the organic solvent collecting tank 21. To collect the mother liquor and the organic solvent for washing in only one organic solvent collecting tank 21 as shown in the figure, it is necessary that the organic solvent used to dissolve the DCDPS and the organic solvent for washing are the same.

For purifying, the organic solvent collected in the organic solvent collecting tank 21 is fed into a distillation column 23. In the distillation column high boilers and low boilers are separated. The low boilers comprise essentially the organic solvent and the high boiler comprises non-crystallized DCDPS and high boiling impurities. The low boiling organic solvent then is fed into an organic solvent storage tank 25.

After washing, the washed DCDPS is withdrawn from the filtration apparatus 19 and fed into a dryer 27. In the dryer the organic solvent is removed from the DCDPS. The dried DCDPS preferably contains less than 400 ppm organic solvent. The dried DCDPS is withdrawn from the dryer 27 as product 29. The organic solvent which is separated from the DCDPS in the dryer by evaporation is withdrawn from the dryer 27 and fed into a condenser 31. To support evaporation and to avoid an oxidation, an inert gas 33, preferably nitrogen, is fed into the dryer 27. The inert gas is withdrawn from the dryer 27 together with the evaporated organic solvent. In the condenser 31, the organic solvent is separated from the inert gas. The condensed organic solvent is fed into the organic solvent storage tank 25 and the inert gas is vented via venting line 35.

To provide a sufficient amount of organic solvent and to replace organic solvent withdrawn from the process for example from the gastight closed vessel 5, the condenser 31 or the distillation column 23, fresh organic solvent 37 can be added into the organic solvent storage tank 25.

From the organic solvent storage tank 25, the organic solvent is supplied to the gastight closed vessel 5 for producing the solution and to the filtration apparatus 19 for washing the DCDPS.

### EXAMPLES

500.4 g crude DCDPS containing 115 g water and containing about 0.24% heptanoic acid and about 240 ppm isomers of 4,4′-DCDPS were suspended into 1385 g methanol. This mixture was heated to a temperature of 100° C. in a closed vessel. The temperature was kept at 100° C. for 2 h and 20 min. Then the pressure in the vessel was reduced and methanol started to evaporate. Evaporation of methanol resulted in crystallization of the DCDPS. The temperature in the vessel was reduced linearly with a rate of 10 Kelvin per hour until a temperature of 10° C. was reached. After this temperature was reached, the vessel was vented until ambient pressure was achieved. The thus obtained mixture of crystallized DCDPS and methanol was filtered in a filter nutsche. By this filtration a wet filter cake which weighted 613,5 g was obtained. The wet filter cake was washed with fresh 400 g methanol. Afterwards, the washed wet filter cake was dried for 5 hours in a Rotavapor® rotary evaporator with a wall temperature of 130° C. The thus obtained product had the following composition:

99,987% 4,4′-DCDPS

120 ppm methanol

90 ppm DCDPS-isomers

20 ppm remaining carboxylic acid.

### LIST OF REFERENCE NUMERALS

1 crude DCDPS

3 organic solvent

5 gastight closed vessel

7 double jacket

9 agitating means

11 condenser

13 vacuum pump

15 line

17 buffer container

19 filtration apparatus

21 organic solvent collecting tank

23 distillation column

25 organic solvent storage tank

27 dryer

29 product

31 condenser

33 inert gas

35 venting line

37 organic solvent

1.-16. (canceled)

17. A process for purifying crude 4,4′-dichlorodiphenyl sulfone comprising:
(a) obtaining a solution which comprises 4,4′-dichlorodiphenyl sulfone, an organic solvent, in which 4,4′-dichlorodiphenyl sulfone has a solubility of 0.5 to 20% at 20° C., and 1 to 30 wt % water based on the amount of 4,4′-dichlorodiphenyl sulfone and water by dissolving the crude 4,4′-dichlorodiphenyl sulfone which optionally contains water in the organic solvent and optionally adding water;
(b) cooling the solution to a temperature below the saturation point of 4,4′-dichloro-diphenyl sulfone to obtain a suspension comprising crystallized 4,4′-dichlorodiphenyl sulfone;
(c) carrying out a solid-liquid separation to obtain residual moisture containing 4,4′-di-chlorodiphenyl sulfone and a mother liquor;
(d) washing the residual moisture containing 4,4′-dichlorodiphenyl sulfone with an organic solvent in which 4,4′-dichlorodiphenyl sulfone has a solubility of 0.5 to 20% at 20° C.;
(e) optionally repeating steps (b) to (d);
(f) drying the 4,4′-dichlorodiphenyl sulfone.

18. The process according to claim 17, wherein the solution in (b) is cooled with a cooling rate of from 10 to 40 K/h.

19. The process according to claim 17, wherein the solution in (b) is cooled to a temperature in the range from 0 to 25° C.

20. The process according to claim 17, wherein for dissolving the crude 4,4′-di-chlorodiphenyl sulfone in the organic solvent in which 4,4′-dichlorodiphenyl sulfone has a solubility of 0.5 to 20% at 20° C., a suspension is formed comprising the crude 4,4′-di-chlorodiphenyl sulfone and the organic solvent in which 4,4′-dichlorodiphenyl sulfone has a solubility of 0.5 to 20% at 20° C. and to heat the suspension to a temperature in the range from 90 to 120° C.

21. The process according to claim 17, wherein cooling (b) comprises:
(i) reducing the pressure of the solution to a pressure at which the organic solvent in which 4,4′-dichlorodiphenyl sulfone has a solubility of 0.5 to 20% at 20° C. starts to evaporate;
(ii) condensing the evaporated organic solvent in which 4,4′-dichlorodiphenyl sulfone has a solubility of 0.5 to 20% at 20° C. by cooling;
(iii) mixing the condensed organic solvent in which 4,4′-dichlorodiphenyl sulfone has a solubility of 0.5 to 20% at 20° C. with the solution to obtain the suspension.

22. The process according to claim 21, wherein the pressure is reduced stepwise or continuously.

23. The process according to claim 21, wherein after completing cooling the pressure is set to ambient pressure.

24. The process according to claim 17, wherein the solid-liquid separation is a filtration.

25. The process according to claim 17, wherein the solid-liquid separation and the washing are carried out in the same apparatus.

26. The process according to claim 17, wherein at least a part of the mother liquor and optionally the organic solvent in which 4,4′-dichlorodiphenyl sulfone has a solubility of 0.5 to 20% at 20° C. used for washing are worked up by distillation.

27. The process according to claim 17, wherein 50 to 100 wt % of the mother liquor and optionally the organic solvent in which 4,4′-dichlorodiphenyl sulfone has a solubility of 0.5 to 20% at 20° C. used for washing are worked up by distillation.

28. The process according to claim 17, wherein the organic solvent in which 4,4′-dichlorodiphenyl sulfone has a solubility of 0.5 to 20% at 20° C. in which the 4,4′-dichlorodiphenyl sulfone is solved and the organic solvent in which 4,4′-dichlorodiphenyl sulfone has a solubility of 0.5 to 20% at 20° C. for washing are the same.

29. The process according to claim 17, wherein the organic solvent in which 4,4′-dichlorodiphenyl sulfone has a solubility of 0.5 to 20% at 20° C. is methanol, ethanol, isopropanol, acetone, methyl tert-butyl ether, acetic acid, toluene, ethyl acetate or monochlorobenzene.

30. The process according to claim 17, wherein the drying (f) is carried out in a contact dryer, wherein the contact dryer preferably is operated with a wall temperature in the range from 105° C. to 140° C.

31. The process according to claim 17, wherein the crude 4,4′-dichlorodiphenyl sulfone comprises n-hexanoic acid, n-heptanoic acid or a mixture thereof which is removed by washing with the organic solvent in which 4,4′-dichlorodiphenyl sulfone has a solubility of 0.5 to 20% at 20° C.

32. Use of a gastight closed vessel for cooling a solution which comprises 4,4′-dichlorodiphenyl sulfone, organic solvent and 1 to 30 wt % water based on the amount of 4,4′-dichlorodiphenyl sulfone and water, by:
(i) reducing the pressure of the solution to a pressure at which the organic solvent in which 4,4′-dichlorodiphenyl sulfone has a solubility of 0.5 to 20% at 20° C. starts to evaporate;
(ii) condensing the evaporated organic solvent in which 4,4′-dichlorodiphenyl sulfone has a solubility of 0.5 to 20% at 20° C. by cooling;
(iii) mixing the condensed organic solvent in which 4,4′-dichlorodiphenyl sulfone has a solubility of 0.5 to 20% at 20° C. with the solution to obtain the suspension.
